(19) United States
(12) Patent Application Publication
(10) Pub. No.: US 2006/0161087 A1
(43) Pub. Date: Jul. 20, 2006

(54) Spinal compression system and methods of use

(75) Inventors: Clayton R. Carter, Logan, UT (US); David M. Carter, Logan, UT (US)

Correspondence Address: WORKMAN NYDEGGER;(F/K/A WORKMAN NYDEGGER & SEELEY), 60 EAST SOUTH TEMPLE, 1000 EAGLE GATE TOWER, SALT LAKE CITY, UT, 84111, US

(73) Assignee: Portal, Inc.

(21) Appl. No.: 11/332,639

(22) Filed: Jan. 13, 2006

Related U.S. Application Data
(60) Provisional application No. 60/644,832, filed on Jan. 17, 2005.

(51) Int. Cl. A61F 5/00; A61F 5/37

(52) U.S. Cl. 602/32; 128/870; 602/33; 602/36

(57) ABSTRACT

A spine compression device for use during diagnostic imaging or diagnosing back pain includes a base having a body side and a bed side. A footplate upstands on the body side of the base and is movably coupled with the base. At least one resilient member has a first end at least indirectly coupled with the footplate. A drive assembly is configured to apply a compressive or tensioning force to the at least one resilient so that the spring applies a force to the footplate, thereby causing the footplate to move relative to the base.

### CROSS-REFERENCE TO RELATED APPLICATIONS

This patent application claims the benefit of provisional patent application Ser. No. 60/644,832, filed Jan. 17, 2005, which is incorporated herein by specific reference.

### BACKGROUND OF THE NENTION

1. The Field of the Invention

The present invention relates to compression systems for loading a spine, joints, and related tissue of a patient for diagnostic imaging or diagnosing spine pain.

2. The Relevant Technology

Traditionally, when a patient is examined using either a conventional magnetic resonance imaging (MRI) unit or a computerized tomography (CT) scan unit, and regularly when a patient is examined using an x-ray unit, the patient is placed in a supine, relaxed and mobile position. Such positioning of the patient compromises the effectiveness of the MRI unit, CT scan unit, and x-ray unit as a diagnostic tool.

For example, a substantial problem associated with placing patients in a relaxed and supine position during an examination of the skeleton, joints and spine using conventional MRI units, CT Scan units, or x-ray units is that the resulting diagnosis is often inaccurate. Studies have shown that the pressure placed on a patient's skeleton, joints and spine while in the relaxed supine position is significantly less than the pressures on the patient's skeleton, joints and spine while the patient is sitting, standing or walking. Therefore, when an MRI unit, CT scan unit or x-ray unit is used to diagnose injuries and disease in a patient's skeleton, joints and spine, their effectiveness as diagnostic instruments is compromised. This occurs because the reduced pressures on the skeleton, joints and spine cannot accurately re-create the conditions existing in the skeleton, joints or spine when the patient is sitting, standing or walking.

By way of example, conventional MRI techniques are often used in the diagnosis of lumbar disc disease or injury. Experience has shown that it is not uncommon to find a disassociation between the severity of the patient's clinical symptoms and evidence of injury or disease shown through MRI imaging. This disassociation can be explained, in part, by the general inability of conventional MRI diagnosis techniques to allow the patient to be imaged while in a variety of positions, including the standing or sitting positions, to vary the intra-discal pressures and alignment of the vertebrae. The relaxed supine position, in which all conventional MRIs of the lumbar spine are performed, is associated with the lowest intra-discal pressure, and is thus not a good position to provoke disc herniation, and is thus not the optimal position for accurate disc herniation diagnosis. This same problem is experienced with the imaging and diagnosis of skeleton, joint and other spine injury or disease.

Attempts have been made to mechanically compress the spine so as to address the above problem. Such attempts, however, have significant shortcomings. For example, many prior art attempts require the patient to be secured to an enlarged, specially designed table or other apparatus. Such tables and apparatus, however, are too large to operate with an MRI unit which has minimal operating space. Such tables and apparatus can also be inconvenient or unusable with some CT and x-ray units. In other attempts, the apparatus comprises materials which compromise or affect the accuracy or operation of an MRI unit. In still other attempts, the apparatus and methods are unable to predictably and controllably exert and maintain pressure on the spine and other areas during the imaging procedures.

Accordingly, what is needed are methods and apparatus for the positioning of a patient's skeleton, joints and spine for imaging with either a conventional MRI unit, CT scan unit, or x-ray unit which allow the skeleton, joints and spine of a patient to be readily and easily placed in an orientation which may assist in the diagnosis of injured or diseased areas of each the skeleton, joints and spine by applying substantially the same pressure on each as they would experience while the patient is sitting, standing or walking.

What is further needed are methods and apparatus which allows a patient to be readily and easily oriented for imaging with either a conventional MRI, CT Scan, or x-ray imaging device in a manner which predictably, controllably, variably and accurately applies a pressure on the skeleton, joints, and spine of the patient during the imaging process.

Methods and apparatus are also needed which may be utilized in connection with either MRI, CT Scan, or x-ray imaging devices of conventional construction for enhanced imaging of a patient and which do not compromise or affect the accuracy or operation of the MRI, CT Scan and x-ray imaging devices.

### BRIEF DESCRIPTION OF THE DRAWINGS

To further clarify the above and other advantages and features of the present invention, a more particular description of the invention will be rendered by reference to specific embodiments thereof which are illustrated in the appended drawings. It is appreciated that these drawings depict only typical embodiments of the invention and are therefore not to be considered limiting of its scope. The invention will be described and explained with additional specificity and detail through the use of the accompanying drawings in which:

FIG. 1 is a perspective view illustrating an embodiment of a spinal compression system;

FIG. 2 is a perspective view of a load biasing assembly of the compression system of FIG. 1;

FIG. 3 is a top exploded view of a support assembly of the load biasing assembly shown in FIG. 2;

FIG. 4 is a bottom exploded view of the support assembly shown in FIG. 2;

FIG. 5 is a top perspective of a base and support plate of the load biasing assembly shown in FIG. 2;

FIG. 6 is an exploded bottom view of the base and a spring assembly of the load biasing assembly;

FIGS. 7 is a perspective view of the drive shafts of the load biasing assembly;

FIG. 8 is a bottom perspective view of the load biasing assembly without a cover plate;

FIG. 9 is a perspective view of a drive assembly for the load biasing; assembly;

FIG. 10 is a perspective view of an alternative embodiment of a harness usable with the spinal compression device of FIG. 1;

FIG. 11 is a perspective view of another alternative embodiment of a harness usable with the spinal compression device of FIG. 1;

FIG. 12 is a perspective view of still another alternative embodiment of a harness usable with the spinal compression device of FIG. 1;

FIG. 13 is a perspective view of an MRI unit having a compression system and RF receiver disposed therein;

FIG. 14 is a perspective view of an alternative embodiment of a compression system;

FIG. 15 is a top perspective view of the load biasing assembly of the compression system shown in FIG. 14;

FIG. 16 is a bottom perspective view of the load biasing assembly shown in FIG. 15 with some of the cover plates removed;

FIG. 17 is a perspective view of an MRI unit having a compression system integrally formed therewith; and

FIG. 18 is a perspective of an alternative embodiment of a compression system configured for engaging the head of the patient.

### DETAILED DESCRIPTION OF EMBODIMENTS OF THE INVENTION

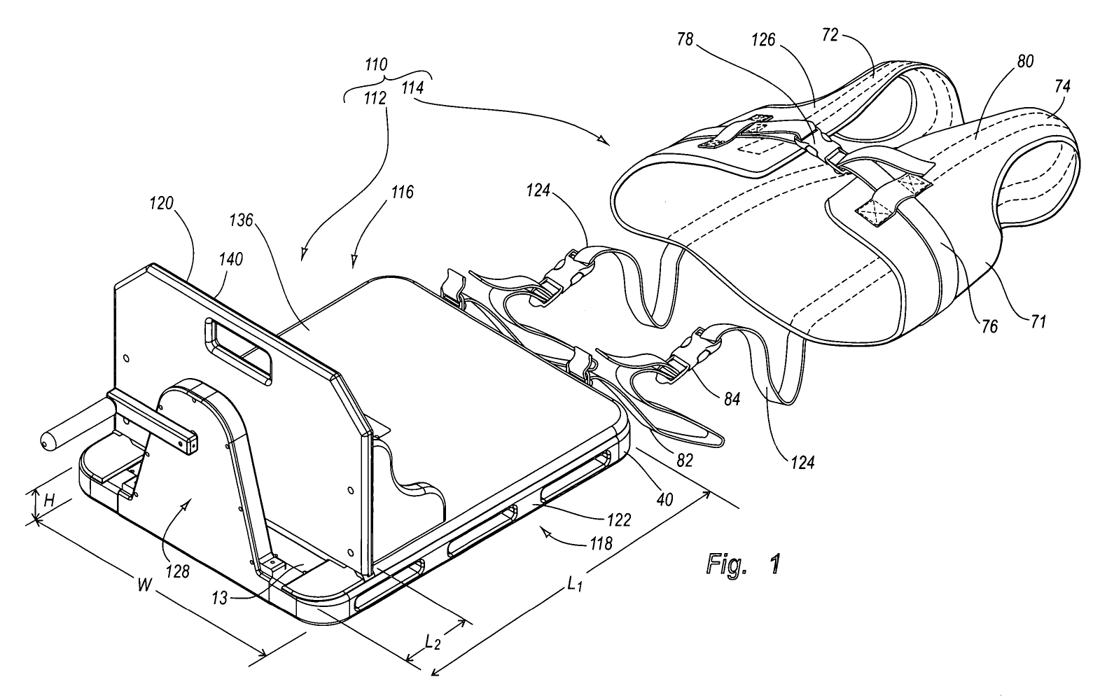

Depicted in FIG. 1 is one embodiment of a compression system 10 incorporating features of the present invention. The compression system 110 is designed to receive a patient in a supine position but can also be used when the patient is in a prone or side position. In general, compression system 110 can load or compress the spine, joints, such as the hip joint, knee joints, and ankles joints, and related soft tissue of a patient so that theses areas of the patient are subject to substantially the same loads or forces that they would be subject to when the patient is in a normal standing, upright position. Many machines which produce diagnostic images are designed to operate with the patient in a supine position. Thus, compression system 10 can be used to obtain diagnostic images of a spine and other areas of a patient while the patient is in the supine position but which accurately depict the spine, joints, and associated bones and/or tissue as if the patient was in a standing upright position.

As used in the specification and appended claims, the term “diagnostic imaging” comprises using systems that produce internal images of a patient without necessarily using invasive procedures on the patient. Examples of “diagnostic imaging” include producing internal images using x-rays, ultrasound, magnetic resonance imaging (MRI), computerized tomography scans (CT scans), fluoroscope, and the like. “Diagnostic image(s)” are images that are produced using “diagnostic imaging.” Diagnostic images can be two or three dimensional images of a patient.

The compression system 110 comprises a load biasing assembly 112 and a restraint assembly 114. The load biasing assembly 112 has a body side 116 and an opposing bed side 118. The body side 116 is oriented such that during operation it receives a lower portion of a patient such as their legs and/or feet. The bed side 118 is oriented so that it can rest on a bed, table, or other substantially flat support structure that receives a patient in the supine position during diagnostic imaging.

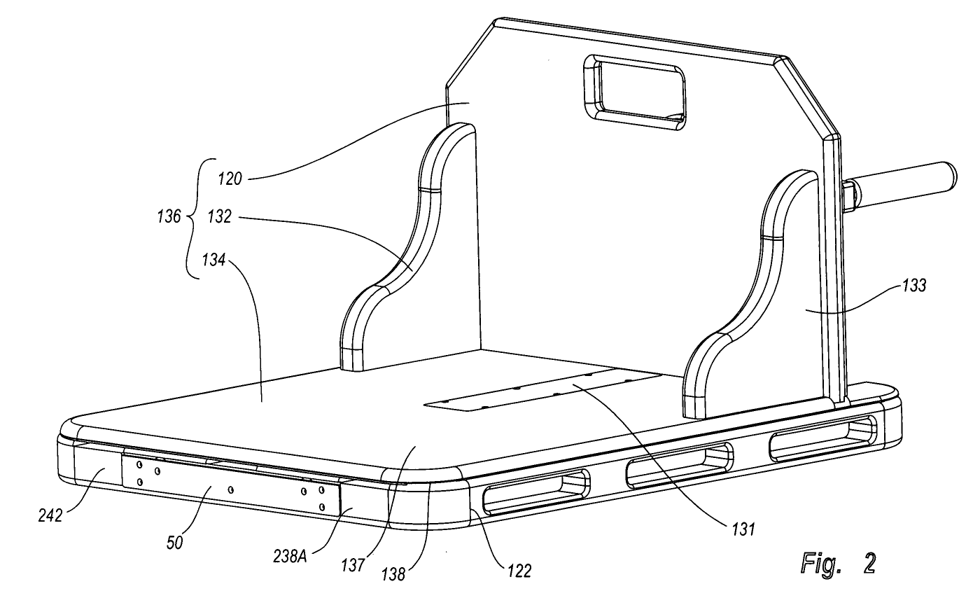

Load biasing assembly 112 generally comprises a base 122, a support assembly 136 which rides on base 122, and a drive assembly 128 which selectively moves the support assembly 136 relative to base 122. As depicted in FIG. 2, the support assembly 136 includes a support plate 134, a foot plate 120 upstanding from the support plate 134, and braces 132 and 133 extending therebetween.

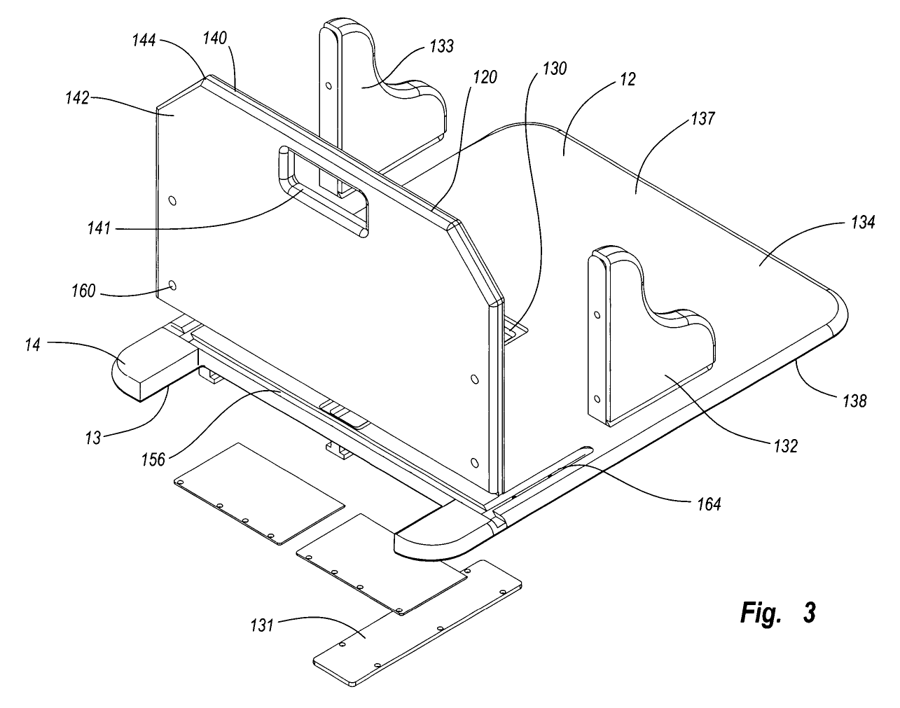

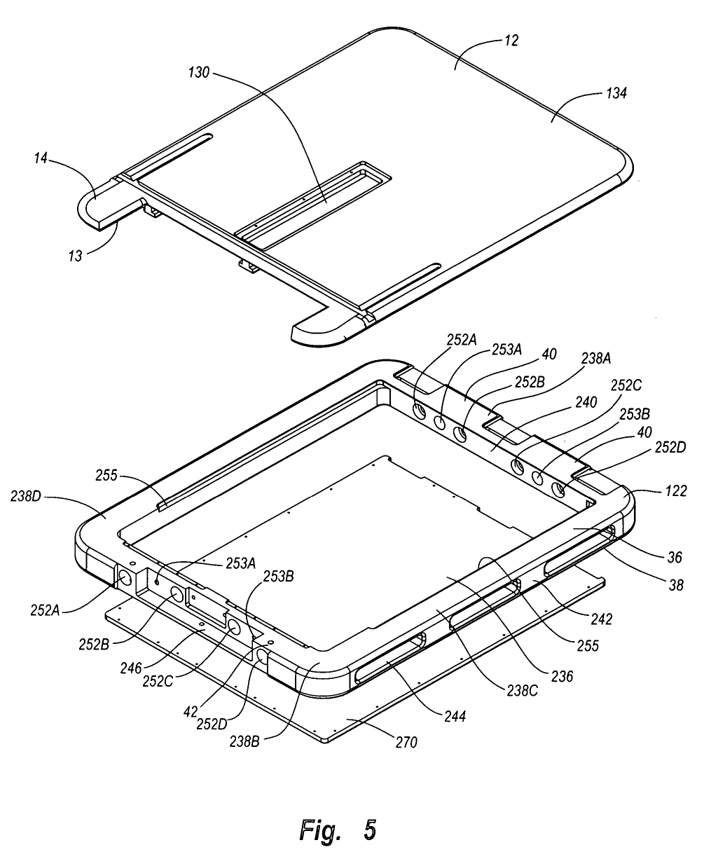

As depicted in FIG. 3, the support plate 134 includes a top surface 137 for receiving a lower portion of the patient such as the legs and/or feet and a bottom surface 138 oriented adjacent to the base 122 (FIG. 2). Top surface 137 and bottom surface 138 each extend between a first end 12 and an opposing second end 14. An elongated notch 13 is centrally formed along second end 14. An elongated slot 130 (FIG. 5) centrally extends from adjacent to notch 13 to a distance toward first end 12. As will be discussed below in greater detail, a transparent cover plate 131 is mounted on support plate 134 so as to cover slot 130.

As shown in FIG. 3, support plate 134 also includes a footplate groove 156 formed on top surface 137 and extending between opposing sides of support plate 134 adjacent to notch 13. A pair of brace grooves 164 intersect with each end of footplate groove 156 and project a distance toward first end 12 on top surface 137.

Footplate 120 includes a front face 140 for receiving the feet of the patient, an opposing back face 142, and a peripheral side 144 extending therebetween. A grip aperture 141 extends through the upper end of foot plate 120 adjacent peripheral side 144. Grip aperture 141 functions to form a handle for lifting load biasing assembly 112. Footplate 120 orthogonally upstands at second end 14 of support plate 134 by being disposed and secured within footplate groove 156.

Braces 132 and 133 extend between and are connected to front face 140 of footplate 120 and top surface 137 of support plate 134 at each side thereof. Corresponding brace grooves 164 can be formed on footplate 120 and support plate 134 to receive the perimeter edge of each brace 132 and 133. Braces 132 and 133 function in part to stabilize and support foot plate 120 relative to support plate 134. Braces 132 and 133 also function as a safety feature so that a foot of the patient cannot unintentionally slip from the footplate 120.

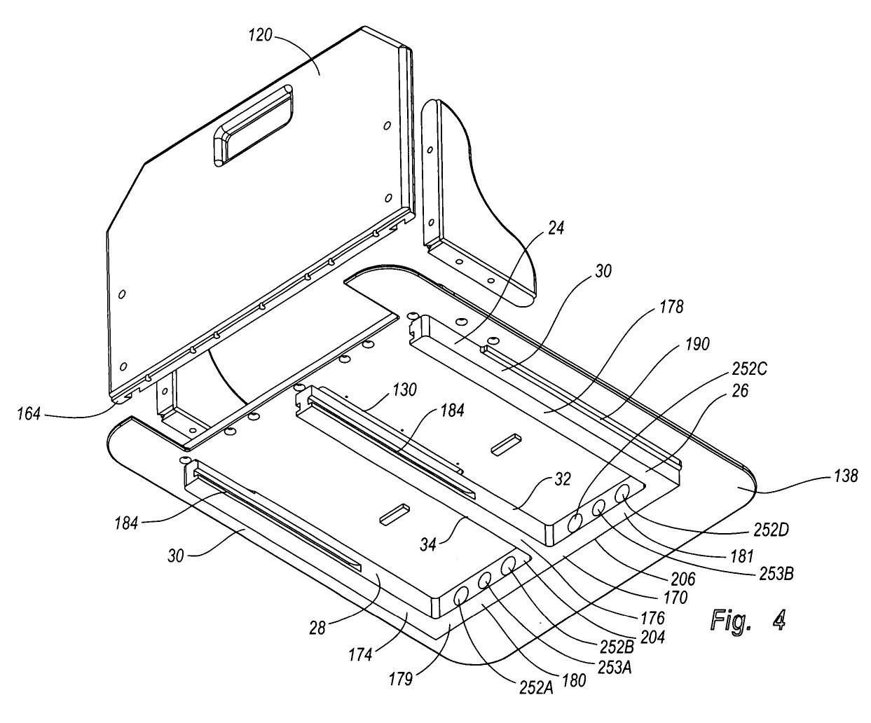

As depicted in FIG. 4, affixed to or integrally formed on bottom surface 138 of support plate 134 is a transfer member 170 having a substantially w-shaped configuration. In the embodiment depicted, transfer member 170 comprises three substantially parallel guide rails that include a first lateral guide rail 174, a center guide rail 176, and a second lateral guide rail 178. Each guide rail has a first end 24 and an opposing second end 26. Guide rails 174 and 178 each have an inside face 28 and a corresponding outside face 30. An elongated channel 184 extends along the length of each inside face 28 at first end 24 while an elongated alignment rail 190 outwardly projects along the length of outside face 30 at second end 26. Central guide rail 176 has opposing side faces 32 and 34 each having an elongated channel 184 extending along the length thereof at first end 24. As will be discussed below in greater detail, a slide rail 190 is positioned at the intersection of outside face 30 of guide rail 174 and support plate 134 and at the intersection of outside face 30 of guide rail 178 and support plate 134.

Each of guide rails 174, 176, and 178 are connected together at second end 26 by as transfer base 179. Transfer base 179 includes base rails 180 and 181. Specifically, guide rails 174 and 176 are connected together at second end 26 by first base rail 180 while guide rails 176 and 178 are connected together at second end 26 by second base rail 181. Extending through base rails 180 and 181 from an inside face 204 to an outside face 206 are guide apertures 252A and B and 252C and D, respectively. Extending through base rail 180 between inside face 204 to an outside face 206 at a location between guide apertures 252A and B is a drive aperture 253A. Likewise, extending through base rail 181 between inside face 204 to an outside face 206 at a location between guide apertures 252C and D is a drive aperture 253B.

As will be apparent from later disclosure, transfer member 170 in part functions as a guide, functions to transfer a load to support plate 134, and functions to stabilize or reinforce support plate 134. It is appreciated that transfer member 170 can have a variety of different configurations. For example, in one alternative central guide rail 176 can be eliminated or shortened. Likewise, guide rails 174, 176, and 178 need not be connected to or even touching transfer base 179. In addition, each of the elements of transfer member 170 could have a variety of different transverse cross sectional configurations. Transfer member 170 can also form a portion of support assembly 136 and can be integrally formed with support plate 134 or separately attached thereto.

Turning to FIG. 5, base 122 has a top surface 36, an opposing bottom surface 38, and a central cavity 236 extending therebetween. Base 122 also has a first end 40 and an opposing second end 42. More particularly, base 122 comprises four peripheral sides 238A-D. Sides 238A and B are disposed at opposing ends 40 and 42 while sides 238C and D extends between sides 238A and B. Each of the peripheral sides 238 includes an interior surface 240 and an exterior surface 242. Interior surfaces 240 cooperate to define an outer boundary of cavity 236.

Centrally recessed on exterior surface 242 of peripheral wall 238B is a drive receiver 246. Extending through peripheral wall 238B between interior surface 240 and exterior surface 242 are four spaced apart guide apertures 252A-D. Guide apertures 252B and C are disposed within drive receiver 246. Also disposed within drive receiver 246 and extending between interior surface 240 and exterior surface 242 are two drive apertures 253A and B. Drive aperture 253A is disposed between guide apertures 252A and B while drive aperture 253B is disposed between guide apertures 252C and D. Extending through peripheral wall 238A between interior surface 240 and exterior surface 242 are corresponding guide apertures 252A-D and drive apertures 253 A and B that are aligned with the corresponding apertures 253A and B on peripheral wall 238B.

A plurality of spaced apart recessed pockets 244 can be formed on the exterior surface 242 of peripheral sides 238C and D so as to form handles which enable an operator to easily grasp, move and/or carry load biasing assembly 112. An alignment channel 255 is recessed on each of peripheral walls 238C and D so as to longitudinally extend along the intersection between interior surface 240 and top surface 36. In the assembled configuration, support plate 134 is positioned over top surface 36 of base 122 so that slide rails 190 (FIG. 4) are received within corresponding alignment channels 255. Alignment channels 255 are longer than slide rails 190 so that support plate 134 and the rest of support assembly 136 can selectively slide along base 122 to the extent that slide rails 190 can move within channels 255.

In one embodiment slide rails 190 are sized so that slide rails 190 fully support support plate 134 on base 122. That is, with slide rails 190 resting in alignment channels 255, a small gap is formed between support plate 134 and base 122. This configuration reduces frictional engagement between support plate 134 and base 122. To further enhance sliding of support plate 134 on base 122, slide rails 190 can be formed from a low friction material such as TEFLON.

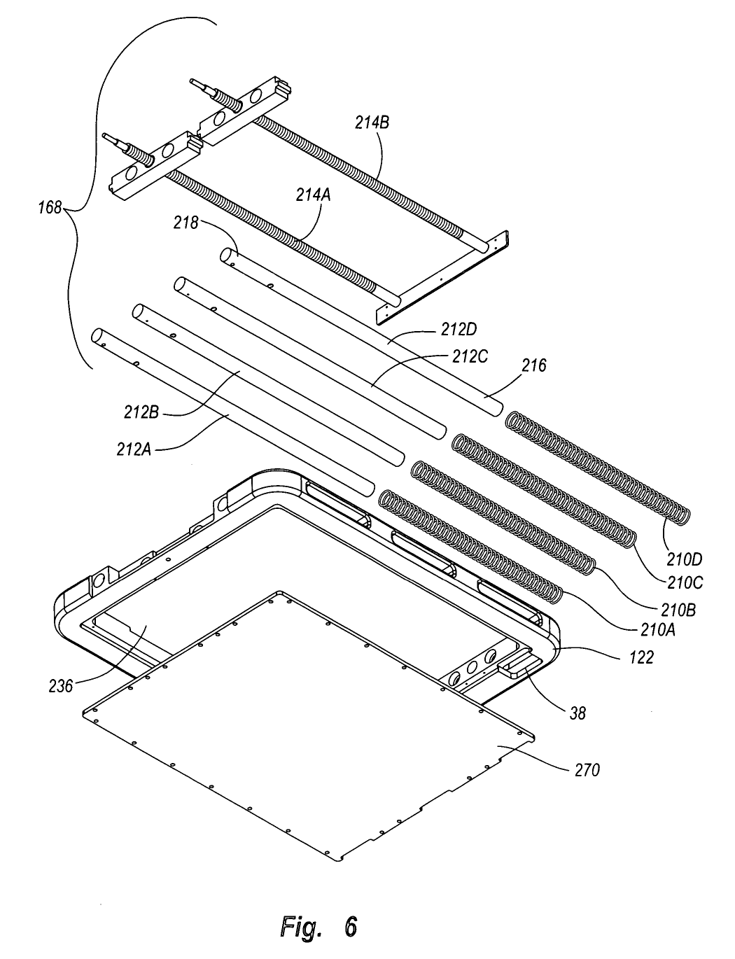

Depicted in FIG. 6, a base plate 270 can be mounted on bottom surface 38 of base 122 so as to cover access to cavity 236 from bottom surface 38. The attachment of base plate 270 to base 122 can be by screws, bolts, welding, adhesive or any other method of connecting.

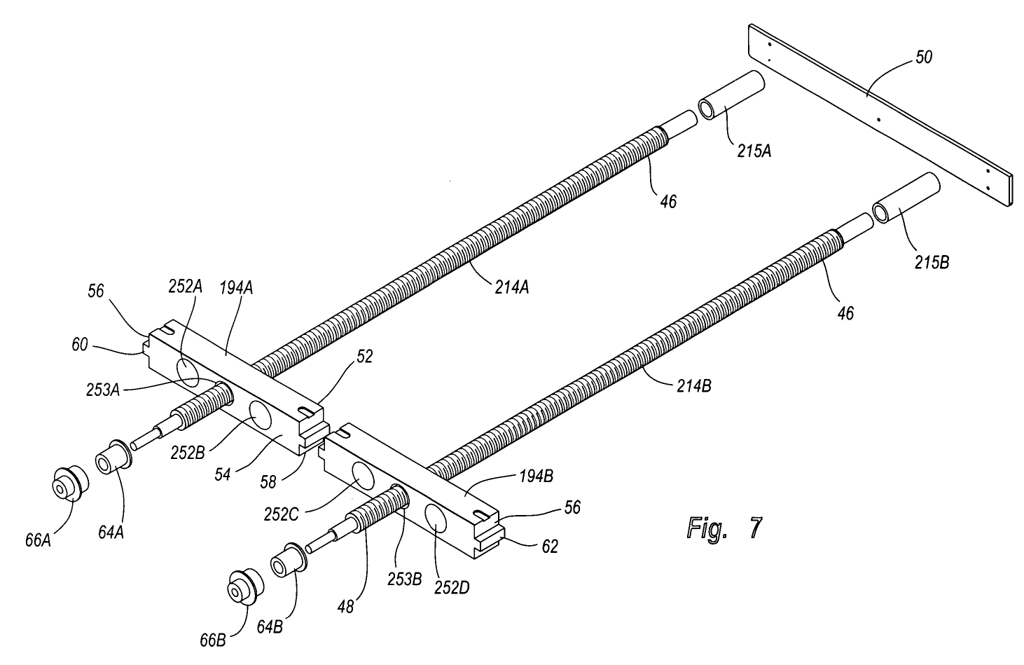

As also depicted in FIG. 6, load biasing assembly 112 also includes a spring loading assembly 168. As depicted in FIG. 7, spring loading assembly 168 includes a pair of threaded drive shafts 214A and B each having a first end 46 and an opposing second end 48. End 46 of each drive shaft 214A and B is configured to be rotatably disposed within a bushing 215A and B, respectively. A retention plate 50 is designed to be mounted on exterior surface 242 of side 238A of base 122 so as to cover the apertures extending therethrough (see FIG. 2). Each second end 48 of drive shafts 214A and B is configured to receive a bushing 64A and B and a spindle 66A and B, respectively.

Threadedly mounted on each drive shaft 214 A and B is a corresponding drive block 194A and B. Each drive block 194A and B has a first side 52 and an opposing second side 54 that each extend between opposing ends 56 and 58. Projecting from end 56 is a drive tooth 60 while projecting from end 58 is a drive tooth 62. Extending through drive block 194A from first side 52 to opposing second side 54 are guide apertures 252A and B. Extending through drive block 194A at a location between guide apertures 252A and B is a threaded drive aperture 253A.

Drive shaft 214A is threaded into drive aperture 253A such that rotation of drive shaft 214A causes drive block 194A to selectively travel along the length of drive shaft 214A based on the direction of rotation. For example, rotation of drive shaft 214A in one direction, such as clockwise, can serve to move drive block 194A toward first end 46 of drive shaft 214A and rotation of the drive shaft 214A in the other direction can serve to move the drive block 194A toward second end 48 of drive shaft 214A. Similarly, extending through drive block 194B from first side 52 to opposing second side 54 are guide apertures 252C and D and a threaded drive aperture 253B. Drive shaft 214B is also threaded into drive aperture 253B.

Returning to FIG. 6, spring loading assembly 168 also includes four guide shafts 212A-D each having a first end 216 and an opposing second end 218. In the embodiment depicted, guide shafts 212A-D have a substantially cylindrical configuration. In alternative embodiments, guide shafts 212A-D can have a non-circular transverse cross section. Encircling each guide shaft 212A-D is a corresponding coiled spring 210A-D.

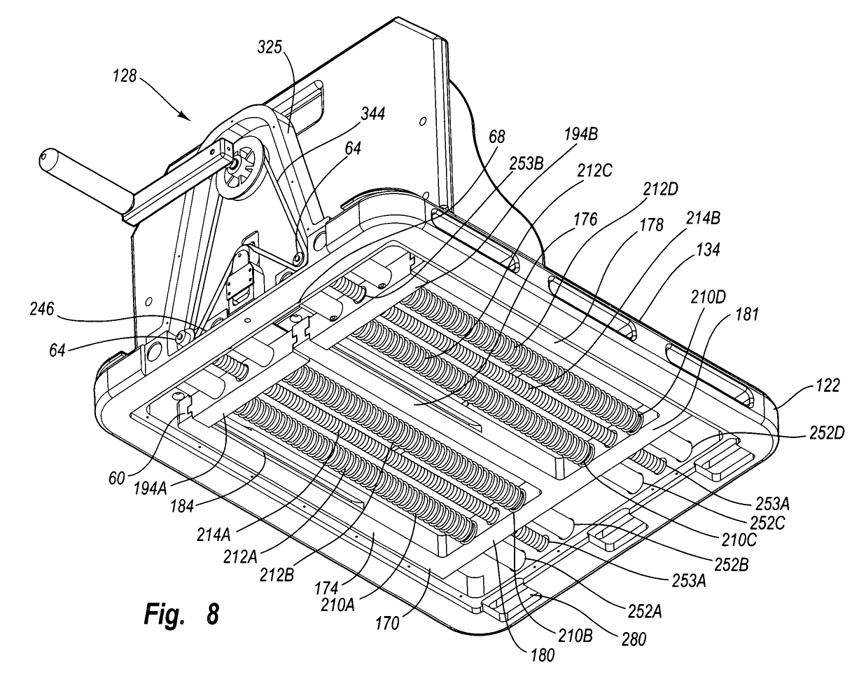

Turning to FIG. 8, during assembly support plate 134 is mounted on base 122 as previously discussed. In this configuration, all corresponding guide apertures 252A-D and drive apertures 253A and B on base 122 and transfer member 170 are longitudinally aligned. Drive block 194A is positioned between guide rails 174 and 176 of transfer member 170 so that teeth 60 and 62 of drive block 194A are slidably received within corresponding channels 184 on guide rails 174 and 176. Similarly, drive block 194B is positioned between guide rails 176 and 178 of transfer member 170 so that teeth 60 and 62 of drive block 194B are slidably received within corresponding channels 184 on guide rails 176 and 178. In this configuration, drive blocks 194A and 194B are free to slide along transfer member 170.

Next, springs 210A and B are positioned between drive block 194A and base rail 180 of transfer member 170 with spring 210A being aligned with guide apertures 252A and spring 210B being aligned with guide apertures 252B. Springs 210A and B have a diameter larger than the diameter of apertures 252A and B so that springs 210A and B directly bias against drive block 194A and base rail 180. Alternatively, springs 210A and B can be smaller and various retention mechanisms, such as a constricting washer, can be used so the springs 210A and B indirectly bias against drive block 194A and base rail 180.

In this position, guide shafts 212A and B are passed through the aligned guide apertures 252A and B, respectively, so that shafts 212A and B also pass through corresponding springs 210A and B. Set screws 68 are passed through base 122 and into each guide shafts 212A and B so as to secure guide shafts 212A and B in place. Other conventional securing techniques can also be used. Guide shafts 212A and B function to facilitate proper aligned travel of drive blocks 194A and B and also prevent bowing of springs 210A and B as they are axially compressed.

The same above process is also used to place guide shafts 212C and D within aligned guide apertures 252C and D so that springs 210C and D encircle guide shafts 212C and D and are disposed between drive block 194B and base rail 181 of transfer member 170.

Once in the above configuration, second end 48 of drive shafts 214A and B are passed through aligned drive apertures 253A and B, respectively, beginning from first end 40 of base 122. As drive shafts 214A and B reach drive apertures 253A and B on drive blocks 194A and B, drive shafts 214A and B are rotated so as to thread into drive apertures 253A and B on drive blocks 194A and B. Second end 48 of each drive shaft 214A and B is advanced so as to pass through drive apertures 253A and B at second end 42 of base 122. It is noted that bushing 215A and B are positioned within drive apertures 253A and B of side 238A of base 122 while bushings 64A and B are positioned within drive apertures 253A and B of side 238B of base 122 so that each drive shaft 214A and B freely spins within corresponding bushings 215A and B and bushings 64A and B. Once drive shafts 214A and B are positioned, spindles 66A and B are coupled at second end 48 of corresponding drive shafts 214A and B using conventional techniques so that spindles 66A and B are disposed within drive receiver 246. Spindles 66A and B are connected so that rotation of spindles 66A and B causes rotation of corresponding drive shafts 214A and B. In this assembly as depicted in FIG. 2, retention plate 50 blocks apertures 252 and 253 on side 238A of base 122 so as to help secure the guide shafts and drive shafts in place.

In alternative embodiments, it is appreciated that any combination of drive shafts, drive blocks, guide shafts and springs can be used. For example, in one embodiment, drive shaft 214B, guide shafts 212C and D and springs 210C and D can be eliminated. In yet other embodiments additional drive shafts, drive blocks, guide shafts and springs can be added. In another embodiment as referenced above, center guide rail 176 can be eliminated and a single drive block used that extends between guide rails 174 and 178.

Furthermore, although springs 210 are depicted as being coiled springs, it is appreciated that springs 210 can have a variety of different configurations. For example, springs 210 can comprise a shaft, tube, or other configuration of resilient elastomeric material, leaf springs, pneumatic springs, or any other spring configuration or material that is capable of producing a resilient biasing force between the drive blocks and the transfer base. As such, the term “spring” as used in the appended claims is intended to encompass all of the above discussed spring embodiments and alternatives thereto.

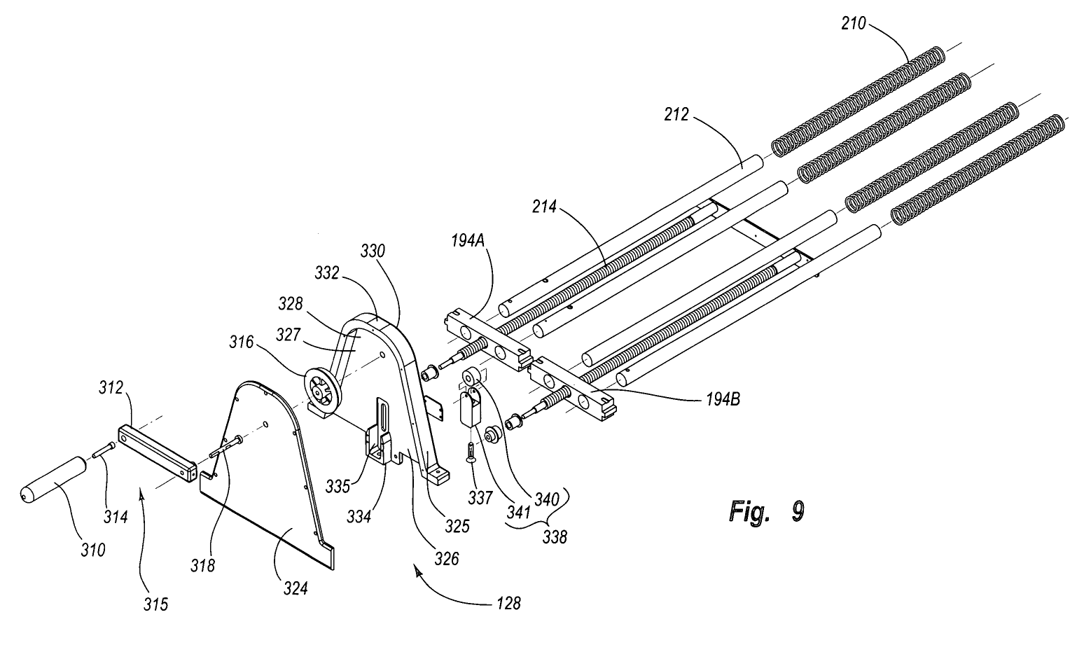

As also depicted in FIG. 8, the present invention includes drive assembly 128 that is used for selectively rotating drive shafts 214A and B. Depicted in FIG. 9, drive assembly 128 comprises a manually operated crank assembly. Specifically, drive assembly 128 comprises a housing 325 having a back plate 326 with an interior surface 328 and an exterior surface 330. A peripheral wall 332 projects forward from around the periphery of interior surface 328 so as to form a cavity 327. Housing 325 is mounted on base 122 so as to be positioned over drive receiver 246 (FIG. 8). A wheel 316 is rotatably mounted on back plate 326 by a pin 318 so as to be disposed within cavity 327.

A tensioner receiver 334 is formed on interior surface 328 of back plate 326 and partially bounds a pocket 335. A belt tensioner 338 is movably disposed within pocket 335. The belt tensioner 338 comprises a tensioning wheel 340 rotatably mounted on a support 341. Support 341 is received within pocket 335 while an adjusting bolt 337 threadedly extends through the floor of the tensioner receiver 334 so as to bias against the floor of support 341. By rotating adjusting bolt 337, tensioning wheel 340 is selectively raised or lowered. A belt 344 (FIG. 8) extends over wheel 316, around each spindle 64 and over tensioning wheel 340. By selectively adjusting the position of belt tensioner 338 as discussed above, belt 344 can be selectively tensioned so that belt 344 properly engages wheel 316 and spindles 64 so as to facilitate proper rotation of drive shafts 214A and B. It is appreciated that any number of conventional and adjustable belt tensioning methods and assembles can be used. In. one embodiment, belt 344, wheel 316, and or spindles 64 can have complementary teeth to further facilitate engagement therebetween.

Removably mounted on housing 325 so as to cover wheel 316, belt 344, and the other components within cavity 327 is a cover plate 324. Pin 318 which extends from wheel 316 passes through cover plate 324. A crank 315 is mounted to pin 318 and is used to manually rotate wheel 316. Specifically, mounted to pin 318 is lever 312. In turn, a handle 310 is mounted to the end of lever 312 by a pin 314.

During operation, an operator rotates handle 310 which in turn rotates wheel 316. Wheel 316 facilitates movement of the belt 344 so that the belt 344 causes simultaneous rotation of both spindles 66. In turn, concurrent rotation of spindles 66 causes concurrent rotation of drive shafts 214A and B which causes drive blocks 194A and B to simultaneously move toward first end 46 of drive shafts 214A and B. Drive blocks 194A and B push against springs 210A-D which in turn push against base rails 180 and 181 of transfer member 170. The application of the force by springs 210A-D against transfer member 170 causes all of support assembly 136 including footplate 120 and support plate 132 to move relative to base 122 in a direction toward first end 40 of base 122.

As will be discussed further below, support assembly 136 continues to move relative to base 122 without significant compression of springs 210A-D until footplate 120 biases against the feet of the user. When this occurs, the force applied by springs 210A-D through support assembly 136 is transferred to the patient. In general, because of the resistance produced by the patient against support assembly 136, further movement of drive blocks 194A and B along drive shafts 214A and B results in springs 210A-D being compressed between drive blocks 194A and B and transfer member 170. As springs 210A-D are compressed, the resilient force produced by springs 210A-D against transfer member 170, and thus against the patient, increases proportionally. Drive blocks 194A and B are continued to be advanced until the patient is loaded with the desired compressive load.

In alternative embodiments, crank 315 can be replaced with a variety of different drive mechanisms for moving wheel 316 and thus belt 344. For example, a drill, pneumatic driver, electric motor or other mechanical driver can be fixed or removably connected to pin 318 or directly to drive shafts 214A and B. In some embodiments, it is desirable not to use a pneumatic pump as the driving mechanism since such system require substantially space and can defeat desired minimal size requirements as discussed below in greater detail.

In another alternative embodiment, it is appreciated that support plate 132 can be rigidly secured to base 122. In this embodiment, transfer member 170 is slidably mounted on the bottom surface of support plate 132 and is coupled directly to footplate 120. As such, movement of transfer member 170 by springs 120 causes movement of footplate 120 relative to support plate 132 and base 122.

In still another alternative embodiment, springs 210A-D can be eliminated. In this embodiment drive blocks 194A and B advance along drive shafts 214A and B until drive blocks 194A and B directly engage against transfer member 170. In turn, transfer member 170 facilitates movement of footplate 120 as previously discussed. In a similar alternative embodiment, both springs 210A-D and drive blocks 194A and B can be eliminated. In this embodiment drive shafts 214A and B can be threaded directly transfer member 170 such that rotation of drive shafts 214A and B facilitates movement of transfer member 170 and foot plate 120.

As discussed above, the amount of force being exerted by footplate 120 against a user is proportional to the amount of compression of springs 210A-D. By knowing the properties of the springs, the amount of force produced by springs 210A-D can be mathematically determined based on the extent of compression. In one embodiment, the extent of compression of springs 210A-D can be detected by an operator looking down through transparent cover plate 131 (FIG. 2) and viewing the relative movement of drive blocks 194A and B. That is, drive blocks 194A and B only move relative to support plate 134 when springs 210A-D are being compressed or expanded. Thus, by placing markings on cover plate 131, support plate 134 and/or the top surface of guide rail 176, a precise displacement of drive blocks 194A and B can be measured so as to convey the corresponding force being exerted by the springs 210A-D. This is the same force which footplate 120 is exerting against the feet of the patient.

In one embodiment, it is desirable to use stainless steel coiled springs having resilient compressive properties in a range between about 3 psi to about 9 psi (2.1-6.2 Newton/square meter) with about 5 psi to about 7 psi (3.4-4.8 Newton/square meter) being more common. Although other types of springs having other properties can be used, spring having the above properties enable restraint assembly 114 to be produced having desired size requirements as discussed herein.

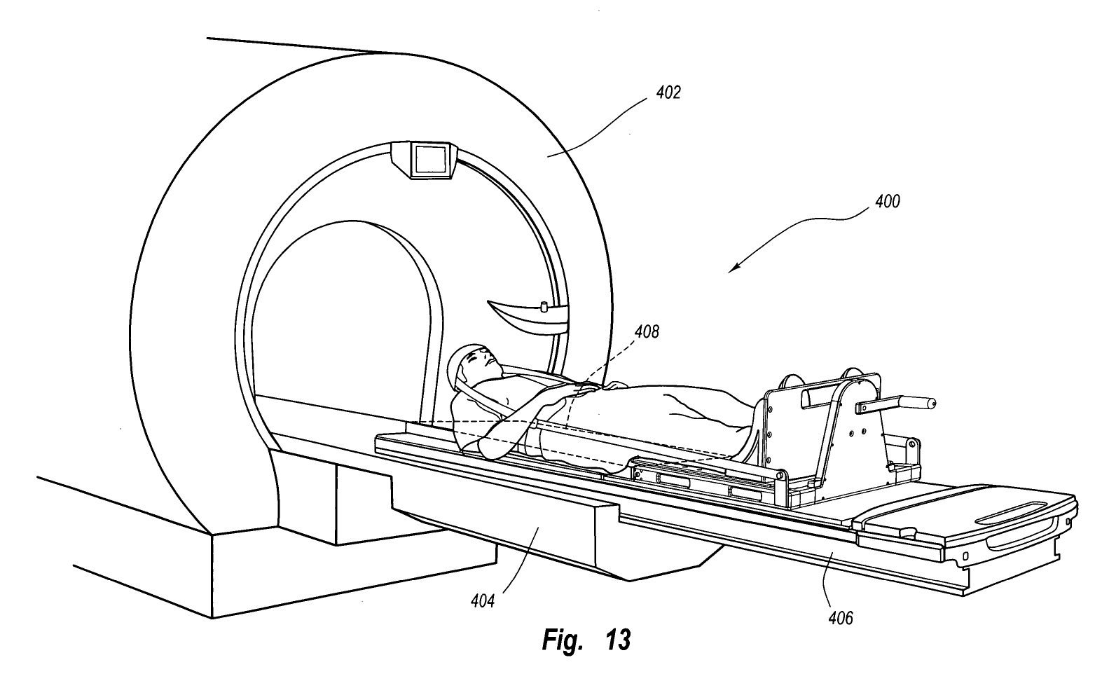

It is appreciated that load biasing assembly 112 is specifically configured and sized so as not to interfere with the diagnostic imaging equipment. That is, compression system 110 is often used on a table that is integrally or specifically designed for use with the machine producing the diagnostic image. For example, depicted in FIG. 13 is one embodiment of an MRI unit 400 that includes a tubular imager 402 having a support 404 connected thereto. A table 406 is mounted on support 404 and is designed to be selectively moved into and out of tubular imager 402. Load biasing assembly 112 is configured so that both load biasing assembly 112 and the patient can be fit on table 406 without interfering with operation of the MRI unit 400.

It is typically desirable that load biasing assembly 112 have a width W (FIG. 1) that is less than or equal to the width of table 406. This helps ensure that table 406 is not obstructed from entering tubular imager 402. As such, width W of load biasing assembly 112 is typically less than 24 inches (60 cm). During imaging of the spine, the patient typically lays on an elongated radio frequency (RF) receiver 408 that is positioned on table 406. Load biasing assembly 112 is typically sized so that it can fit between the RF receiver 408 and the end of table 406. In one embodiment, base 122 of load biasing assembly 112 has a total length L₁ (FIG. 1) in a range between about 30 cm to about 90 cm with about 45 cm to about 75 cm being more common. Load biasing assembly 112 also has a length L₂ extending between front face 140 of foot plate 120 and the back end face of base 122 that is typically less than 15 cm, more preferably less than 12 cm, and most preferably less than 10 cm. Finally, load biasing assembly 112 also has a height H extending from the bottom surface of base 122 to the top surface of support plate 134. Height H is typically substantially the same height as RF receiveir 408 so that when the patient is lying on the RF receiver 408 with his/her legs on the support plate 134, the patient is substantially horizontal. In one embodiment, Height H is in a range between about 1 cm to about 10 cm with about 2 cm to about 6 cm being more common. In alternative embodiments, other dimensions can also be used for the above discussed measurements.

Returning to FIG. 1, restraint assembly 114 can come in a variety of different configurations and is designed to engage the superior end of the patient such as the thorax or head of the patient. In the embodiment depicted restraint assembly 114 generally. comprises a harness 126, which is one for of a restraint, and flexible straps 124. The straps 124 extend between harness 126 and base 122. The restraint assembly 114 is specifically configured to engage the thorax of a patient so as to restrain the patient against the load applied by the load biasing assembly 112. Specifically, the restraint assembly 114 is configured to engage the thorax of a patient so that when footplate 120 pushes against the feet of the patient, the spine of the patient is compressed between the harness 126 and the footplate 120. In addition to the spine, various joints such as the hip joint, knee joints, and ankle joints are also compressed. In addition, various soft tissues related to the spine, such as the vertebral discs, and the joints are also compressed. Compression system 110 can thus be used for obtaining diagnostic images of each of these compressed or loaded regions of the patient.

Compression of the patient is typically accomplished by using a harness that extends over the shoulders of the patient. In alternative embodiments, however, the harness need not extend over the shoulders but could merely constrict around the torso of the patient. In the embodiment depicted, harness 126 comprises a vest 71 having should loops 72 and 74. A chest strap 76 encircles vest 71 and is coupled by an adjustable buckle 78. Each strap 124 has a first end 80 coupled with vest 71 and extending over a shoulder loop 72, 74 and an opposing second end 82 coupled with first end 40 of base 122. Adjustable buckles 84 are disposed between first end 80 and second end 82 so that the length of each strap 124 can be adjusted. In this regard, each strap 124 includes a first strap section and a second strap section that are coupled together by the buckle 84. In this configuration harness 126 can be easily donned by the patient and the harness 126 will securely engage the thorax of the patient.

With reference to FIGS. 1 and 8, the present invention also includes means for attaching the restraint assembly 114 to the load biasing assembly 112. By way of example and not limitation, the base 122 include a plurality of strap receivers 280 that couple with the end of straps 124. The strap receivers 280 are recessed on the bed side 118 of the base 122. Alternatively, the strap receivers 280 can extend from the bed side 118 to either the exterior wall 242 or the body side 116 of the base 122. Other configurations and positions of strap receivers 280 can also be employed. For example, the straps 124 can be attached to the base 122 by a bolts, rivets, screws, adhesive, press fit, or the like.

During use, the patient dons the harness 126 and adjusts the straps 124 so that the harness 126 is properly fitting and straps 124 are properly aligned. It is appreciated that a variety of different sizes of restraint assemblies 114 can be used with a single load biasing assembly 112 to provide proper fit to each patient. Harness 126 is typically donned while the patient is standing and harness 126 is disconnected from load biasing assembly 112. Alternatively, harness 126 can be donned while the patient is in the supine position and connected to load biasing assembly 112. In either event, once harness 126 is donned and the patient is in a supine position, the feet of the patient are placed against the footplate 120 and straps 124 are adjusted so as to remove all slack between load biasing assembly 112 and harness 126.

The operator then manipulates load biasing assembly 112 as discussed above so that footplate 120 moves toward harness 126. In so doing, opposing forces applied between the shoulders of the patient and the feet of the patient cause the spine and other skeletal features and soft tissue of the patient to compress. The footplate 120 is continued to be advanced until the desired compressive force is applied. In one embodiment, compression system 110 is able to apply loads greater than 150 pounds (68 kg) and typically greater than 200 pounds (91 kg). Compression system 110 can also be designed for other load bearing capacities. It is appreciated that the amount of load to be applied may vary for each person and is in part subject to the size of the person and the intended use for compression system 110. In one embodiment, the compressive load applied to the patient is typically in a range between about 40% to about 60% of the patient's weight and is preferably between about 45% to about 55% of the patient weight. Under this applied load, the patient is then subject to diagnostic imaging.

It is noted that after the compressive force is applied, the patient will continue to compress over a period of time during the diagnostic imaging. As a result of this compressing or shrinking of the patient, the original compressive force applied to the patient will slightly decrease during the diagnostic imaging. Significant drops in the compressive force can be undesirable in that the spine and other loaded areas will not reflect the desired loading. By using springs 210A-D to produce a resilient force against the support assembly 136, the amount of compression force on the patient lost due to the continued compressing of the patient is minimized. That is, even if the springs 210A-D are relaxed a small extent due to the continued compression of the patient, the majority of the resilient spring force is still being applied. In contrast, if a static force is applied, any change in the height of the patient could have a substantial change in the amount of force being applied.

However, in the above discussed alternative embodiments where springs are not used, various control systems can be used to continually maintain the desired load on the patient as the patient shrinks. For example, an electrical motor can be mechanically coupled with drive shafts 214A and B and be electrically coupled with a load sensor measuring the compressive load on the patient. As the initial load decreases due to patient shrinkage, a processor senses the drop in load via the load sensor and activates the motor so that the load is increased to the desired level. As a result, the compression load on the patient can be continuously and automatically adjusted during the imaging process. It is also appreciated that the above discussed adjusting system can also be used in conjunction with the use of the springs.

To help account for the gradual compression of the patient, the patient is typically initially loaded with a compressive force that this slightly larger than the desired compression force. The patient then compresses slightly during the time between the initial loading of the compression force on the patient and taking the image of the patient. As a result, the patient is subject to substantially the desired compression force at the time of imaging.

In one embodiment, springs 210A-D can be pre-compressed between drive blocks 194A and B and transfer member 170 at the time of manufacture of load biasing assembly 112. By pre-compressing springs 210A-D the required travel of drive blocks 194A and B can be reduced, thereby saving time in the loading process and reducing the required size for load biasing assembly 112. For example, by pre-compressing springs 210A-D to a load of 50 pounds, this load is directly transferred to the patient when footplate 120 initially biases against the feet of the patient. Thus, the gradual compression of springs 210A-D to produce the first 50 pounds of loading is avoided. In one embodiment, springs 210A-D are pre-compressed to a load in a range between about 30 pounds to about 70 pounds (14-32 kg) with about 40 pounds to about 60 pounds (18-27 kg) being more common. Other pre-compression amounts can also be used.

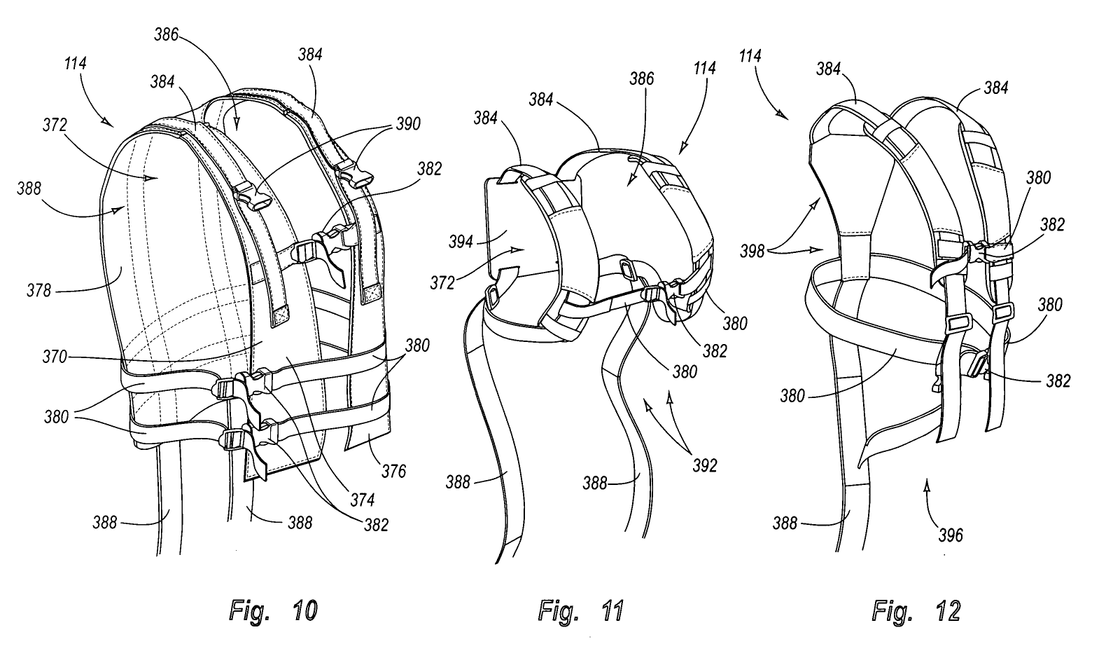

It is appreciated that restraint assembly 114 and straps 124 can come in a variety of different configurations and orientations. For example, FIG. 10 depicts a restraint assembly 114 that includes a vest 370. The vest 370 is configured to be donned by a patient similar to an ordinary jacket or vest. As such, the vest 370 includes arm opening 372 to receive the arms of the patient. Also, the vest includes a right front panel 374, a left front panel 376, and back panel 378 that cooperate to fit around the torso of the patient. Additionally, the vest 370 includes chest straps 380 that can fasten the right front panel 374 to the left front panel 376. More particularly, the chest straps 380 can be fastened together with buckles 382. In one embodiment, the chest straps 380 can extend around the back panel 378 so that the vest 370 can be restrained about the torso or chest of the patient. The vest material can include, for example, neoprene, rubbers, fabrics, and the like.

Additionally, the vest 370 includes shoulder straps 384 that loop over the shoulder portion 386 of the vest 370 from the right front panel 374 to the back panel 378 as well as from the left front panel 376 to the back panel 378. Additionally, it may be preferable for the shoulder straps 384 to be more media and nearer to a neck opening 386. This can position the shoulder straps 384 closer to the neck of the patient as opposed to being more lateral and closer to the arm opening 372 and the arms of the patient. This orientation can provide for more force being applied by the shoulder straps 384 to the torso of the patient without deforming the shoulders of the patient towards their feet. Furthermore, this configuration has been found more comfortable to the patient during loading.

The vest 370 can also include longitudinal straps 388, which can traverse the length of the back panel 378. Additionally, the longitudinal straps can be coupled with longitudinal strap buckles 390 positioned on the right front panel 374 and the left front panel 376. As such, the longitudinal straps 388 can either engage with the load biasing assembly 112 (FIG. 1) or the straps 124 of the restraint assembly 114.

With reference now to FIG. 11, another embodiment of the restraint assembly 114 is illustrated. The restraint assembly 114 is comprised of a series of straps 392 and a back plate 394. The series of straps 392 can include longitudinal straps 388 that traverse the length of the patient, and can optionally be coupled with the back plate 394. Also, the series of straps 392 can include chest straps 380 that can be oriented around the torso or chest of the patient. The chest straps 380 can be fastened together by buckles 382, so that the chest straps 380 can cooperate to restrain the torso or the chest of the patient within the restraint assembly 114. Additionally, the series of straps 392 can include shoulder straps 384 that loop over and around the shoulders of the patient so that a compressive load applied to the feet of the patient can be transferred so as to compress the patient from their shoulder to their feet.

The back plate 394 can serve as a center back piece so that some of the straps (e.g., shoulder straps 384 and longitudinal straps 388) in the series of straps 392 can be coupled with back plate so that the proper restraining and compressive forces can be applied to the patient. Additionally, the straps cooperate to form an arm opening 372 and a neck opening 386. Also, the longitudinal straps 388 can either engage with the load biasing assembly 112 (FIG. 1) or the straps 124 of the restraint assembly 114.

With reference now to FIG. 12, restraint assembly 114 is comprised of strap system 396. The strap system 396 can include chest straps 380, shoulder straps 384, and longitudinal straps 388. Similar to other embodiments of restraint assemblies 114, the chest straps 380 can be wrapped around the torso or chest of the patient to provide a restraint thereto. Also, the shoulder straps 384 can be looped over the shoulders of the patient and connected to the chest straps 380. Further, the longitudinal straps 388 can traverse from the shoulder straps 384 and/or the chest straps 380 along the length of the body of the patient. Alternately, the longitudinal straps 388, shoulder straps 384, and chest straps 380 can all be coupled with a back member 398. The back member 398 can be made of a fabric, neoprene, plastic or the like. Moreover, the longitudinal straps 388 can either engage with the load biasing assembly 112 (FIG. 1) or the straps 124 of the restraint assembly 114. It is appreciated that the various strap systems, harnesses, vests, and other embodiments of the restraint assembly are all examples of restraints that can be used to engage the superior end of the patient.

At least a majority of the components of the compression system 110 are typically made from nonmagnetic materials. As used in the specification and appended claims, the term “nonmagnetic” or similar terms is meant to refer to materials that are not magnetic and do not respond to or interfere with a magnet or a magnetic field. This is because some of the diagnostic imaging equipment, such as an MRI unit, produces an extremely strong magnetic field. If compression system 110 was made from magnetic materials, compression system 110 could be drawn to the magnetic field which would be a potential danger to the patient and/or the magnetic material could interfere with the image produced.

Examples of nonmagnetic materials which compression system 110 can be made of include fabrics, resins, polymers and nonmagnetic metal such as titanium, brass, bronze, low carbon stainless steel such as 316 stainless steel, aluminum or the like. It is appreciated, however, that small amounts of magnetic materials may be used in compression system 110 to the extent that they do not interfere with the safety, operation, or imaging of the system. In selecting the materials, however, weight and strength are also considerations. That is, the materials must be selected so that compression system 110 can withstand the applied loads without failure while minimizing weight so that compression system 110 can be easily moved.

In one embodiment, support assembly 136 and base 122 are made from an acetal plastic such as Acetron GP produced by Quadrant EPP. Acetron GP is a stress free acetal plastic that produces minimal deformation during processing. As such, parts can be made having close tolerances. Guide rods 212 are typically made of titanium while springs 210 are comprised of 316 stainless steel. Finally, drive shafts 214 and drive blocks 194 are typically comprised of brass. The various parts can also be made of other materials. It is also appreciated that the harness, straps and buckles of restraint assembly 114 are also made of nonmagnetic materials. Examples of such materials include natural and synthetic fabrics and webbing, rubbers, elastomeric materials such a Spandex, foam rubbers, neoprenes, and the like. The buckles are typically comprised of plastic. It is appreciated that the various components of compression system 110 can be connected together by adhesive, stitching, welding, rivets, screws, bolts or other conventional fasteners.

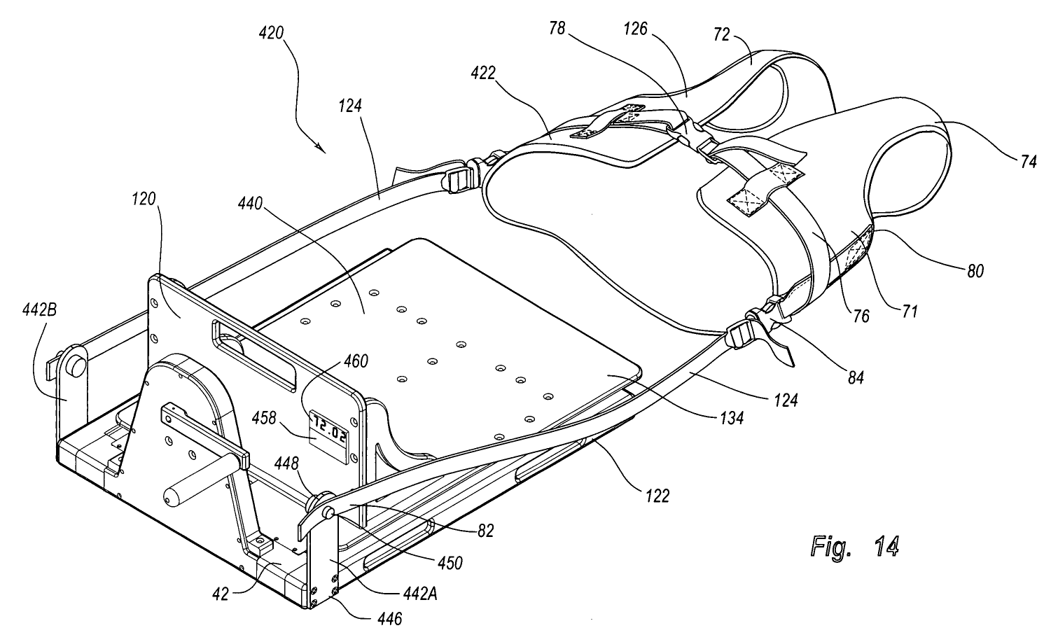

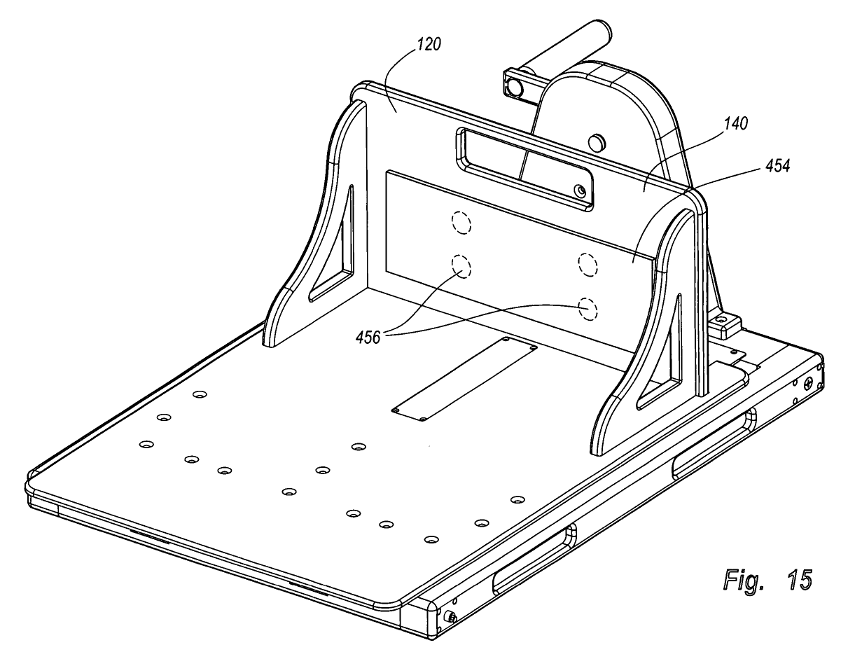

Depicted in FIG. 14 is an alternative embodiment of a compression system 420. Like elements between compression system 420 and compression system 110 are identified by like reference characters. Compression system 420 includes harness 126 previously discussed having opposing sides 422 and 424. In contrast to compression system 110 wherein straps extended from the back of harness 126, a first end 80 of a corresponding strap 124 is connected to a corresponding side 422 or 424 of harness 126. Furthermore, the buckle 84 that is disposed along each strap 124 is positioned adjacent to harness 126. This configuration has a number of benefits. For example, as a result of having buckle 84 positioned adjacent to harness 126, a patient can don harness 126 at any desired time or location and then freely move around without risk of tripping over straps 124. Furthermore, by connecting straps 124 to the side of harness 126, it is easier for the operator to connect the buckles and pull on the tightening strap for removing all slack without the patient or imaging apparatus obstructing the procedure.

Compression system 420 further comprises a load biasing assembly 440. Load biasing assembly 440 is substantially identical to load biasing assembly 112 as previously discussed except for a few modifications. For example, upstanding from second end 42 of base 122 on opposing sides of base 122 are a pair of mounting arms 442A and B. Each mounting arm 442 has a first end 446 connected to base 122 and an opposing second end 448 upstanding from base 122 so as to be disposed above support plate 134. Second end 82 of each strap 124 is connected to second end 448 of a corresponding mounting arm 442. Although not required, in the embodiment depicted strap 124 is pivotally connected to mounting arm 442 by using a fastener 450 such as a bolt or rivet that extends through strap 124 and mounting arm 442. Other fastening techniques can also be used.

As a result of connecting straps 124 to second end 448 of mounting arms 442, during operation when straps 124 are tensioned, mounting arms 442 function as a cantilever which works to force first end 40 of base 122 down against the table of the imaging apparatus. This configuration helps ensure that the load biasing assembly does not interfere with the patient or the imaging apparatus. For example, this configuration helps ensure that first end 40 of base 122 does not raise up during tensioning of straps 124 which could cause the patient's knees to bend or buckle.

It is appreciated that mounting arms 442A and B can be positioned at a variety of locations to achieve the desired objective. For example, mounting arms 442 can be positioned at a variety of locations along the sides of base 122 so long as they are spaced back a distance from first end 40. Furthermore, mounting arms 442 can have a variety of different configurations and be mounted or integrally formed with base 122 in a variety of different ways so long as at least a portion thereof upward projects from base 122.

Turning to FIG. 15, mounted on front face 140 of foot plate 120 is a pressure plate 454. Pressure plate 454 is connected to foot plate 120 by a plurality of spaced apart. load cells 456. As depicted in FIG. 13, load cells 456 are electronically coupled with a processor 458 and an LCD display 460. During operation, the patient places their feet against pressure plate 454. The load applied by the patient on pressure plate 454 is measured by the load cells 456 and an average by the load cells 454 is determined by processor 458 and then displayed on display 460. As a result, an operator can easily determine the load applied to the patient by viewing display 460.

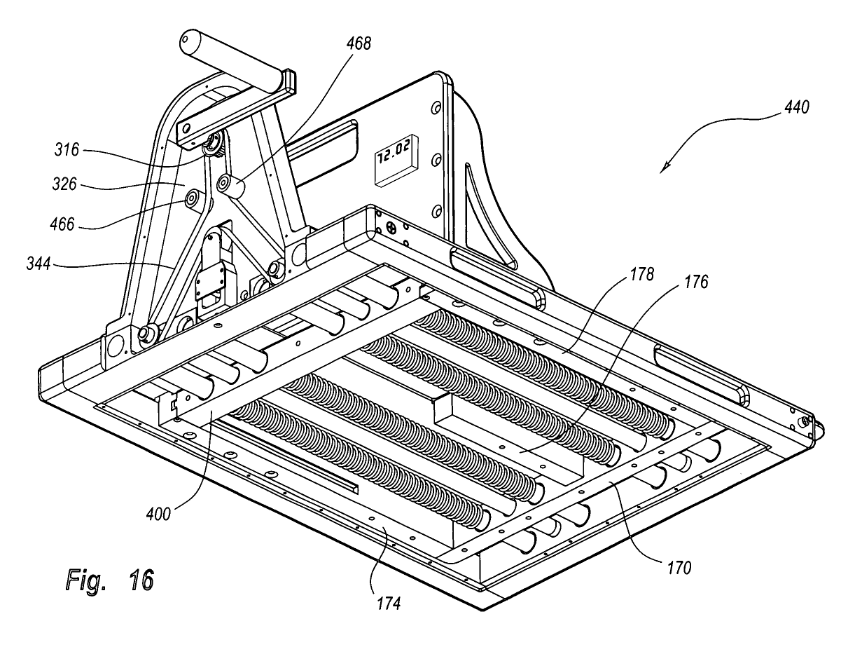

Turning to FIG. 16, in this embodiment, transfer member 170 has been modified so as to shorten central guide rail 176. The two prior drive plates 194A and B have now been replaced with a single drive plate 460 that extends between outside guide rails 174 and 178. It is also noted that additional tensioning wheels 466 and 468 have been mounted on back plate 326 with belt 344 traveling over wheels 466 and 468. Wheels 466 and 468 are positioned so as to help ensure secure engagement between belt 344 and wheel 316 that is driven by the crank.

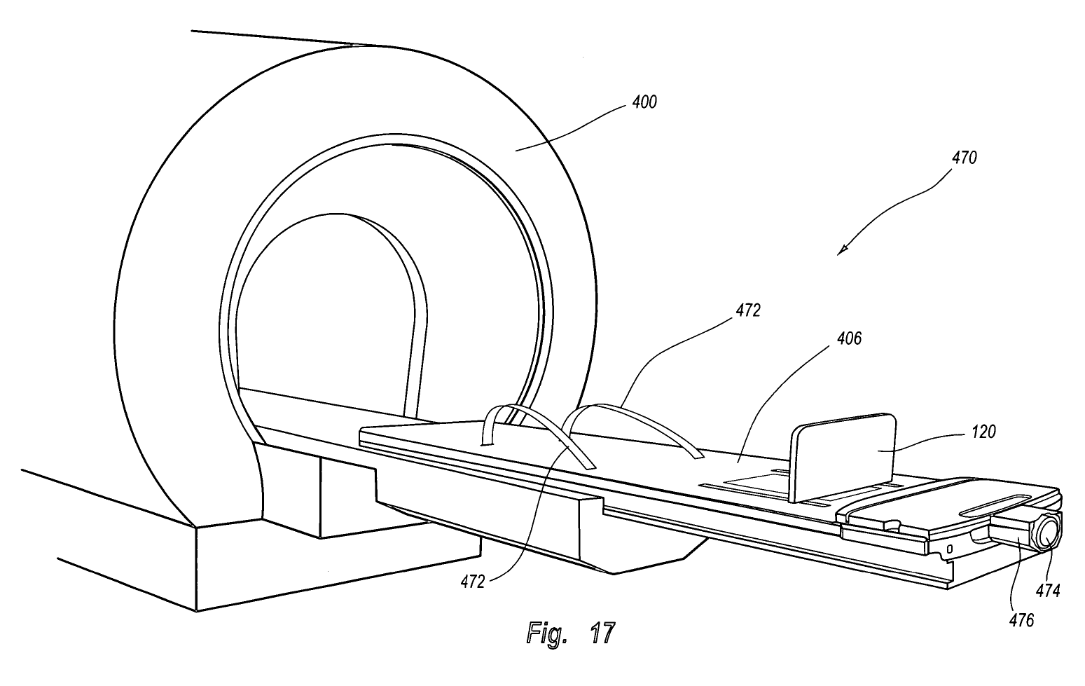

Depicted in FIG. 17 is another embodiment of a compression system 470 incorporating features of the present invention. In this embodiment, compression system is integrally formed as part of MRI unit 400. The base for compression system 470 comprises table 406. Footplate 120 is upstanding from a top surface of table 406 and can selectively move along table 406. The springs, rods, and other mechanisms that facilitate movement of footplate 120 can be disposed within or below table 406. Previously discussed support plate 134 can be eliminated from the present embodiment or can also be mounted on the top surface of table 406.

Compression system 470 further comprises restraints 472 that are mounted directly to table 406 and are adapted to engage the superior end of a patient. In the present embodiment, restraints 472 comprise straps that can pass over the shoulders of the patient. In alternative embodiments, restraints 472 can comprise a harness, vest, webbing, and other similar devices that are connected to table 406 and that can engage the thorax of the patient. In still other embodiments, posts or braces can be directly connected to table 406 against which the shoulders of the patient bias. In yet other embodiments, although foot plate 120 can be moveable coupled directly with table 406, the previously discussed harnesses and straps also can continue to be used with the present embodiment. Specifically, one end of the straps can be connected to the table while an opposing end of the straps can be removably connected to the harness which is not otherwise connected to table 406. The straps can be adjusted to remove the slack once the patient is reclined on the table with their feet biased against foot plate 120.

In contrast to the crank assembly that was previously discussed for use in moving foot plate 120, in the present embodiment an electrical motor 474 is mounted to table 406 and is coupled with previously discussed drive shafts 214. Motor 474 facilitates rotation of drive shafts 214 which in turn facilitate movement of foot plate 120. Motor 474 can be manually operated or coupled with the previously discussed load cells and processor so that the motor can automatically operate to apply and maintain the desired load. Motor 474 is disposed within a housing 476. Housing 476 is comprised of a shielding material such as lead. Housing 476 thus shields motor 474 from the magnetic force produced by MRI unit 400. In an alternative embodiment, motor 474 can be made of a substantially non-magnetic material such as ceramic. Although compression system 470 is shown as being integrally formed as part of MRI unit 400, compression system 470 can also be incorporated into tables that are designed for other diagnostic imaging equipment such as an x-ray unit, CT scan unit, or the like.

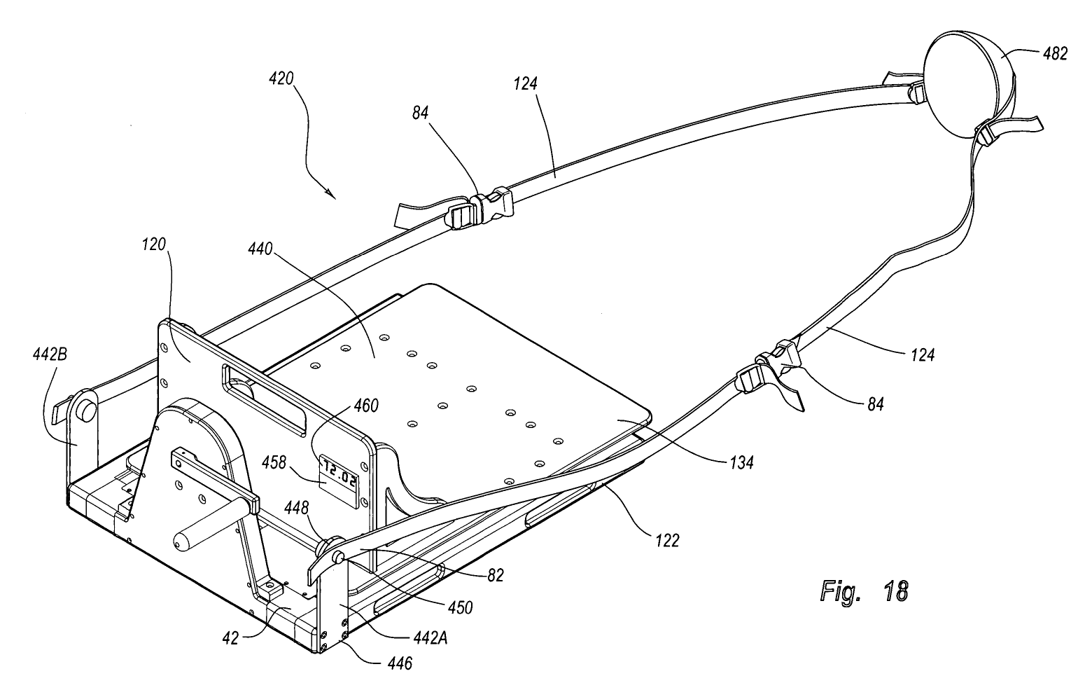

Depicted in FIG. 18 is another alternative embodiment of a compression system 480 incorporating features of the present invention. Compression system 480 is substantially identical to previously discussed compression system 420 except that harness 126 has been replaced with a head restraint 482 which is another example of a restraint for engaging the superior end of the patient. Head restraint 482 can comprise a cap, helmet, or any other type of structure that can engage the head of a patient. Straps 124 engage opposing sides of head restraint 482. During operation, the patient is again placed in a supine position with their feet resting against footplate 120. Head restraint 482 is engaged with the head of the patient and slack is removed from the straps. During application of the compression force, the cervical spine, in addition to the thoracic and lumbar spine, are compressed so that diagnostic imaging can be taken at each of these different spine regions.

In the previous discussions, various compression systems have been discussed primarily in association with diagnostic imaging of the spine. It is also appreciated that the various compression systems of the present invention can also be used in association with diagnosing back pain caused by joint and/or disk degeneration, trauma, or the like in the spine. As part of this diagnoses process, a catheter is implanted within the patient so that a pain relieving agent, such as an anesthetic or steroid, can be delivered to a previously identified location on the spine that is believed to be causing pain. For example, the catheter can be directed to a degenerated disc. The catheter can be implanted under fluoroscopy. It is also appreciated that a needle or other delivery mechanism can also be used to deliver the pain relieving agent to a desired location. For example, the pain relieving agent can also be injected into a facet joint or epidural space.

Either prior to, concurrently with, or following implanting of the catheter or making the injection, the patient is coupled with a compression system such as those disclosed herein. During this procedure, the patient. can be in a supine, prone or side position. The pain relieving agent is then delivered to the intended location on the spine either prior to, concurrently with, or subsequent to loading of the spine using the compression system. In some procedures, it can also be helpful to deliver a saline solution or a contrasting agent, such as a radiopaque contrast, to the location on the spine. If the previously experienced pain is relieved as a result of the pain relieving agent following application of the compression load on the spine, the identified location can be deduced as the source of the pain. If not, other locations can be tested in a similar manner to find the source of the pain. Once the location is determined, isolated treatments or various surgical procedures can be performed to control or remedy the pain.

In accordance with the spinal compression systems and devices of the present invention, a spinal compression device can include a base member that is configured to be positioned under the legs of the patient when the patient is in the supine position. Additionally, the spinal compression device includes a footplate or support assembly that is movably coupled with the base member. As such, embodiments of the present invention include a means for selectively moving the footplate relative to the base. This allows for the base to be positioned under the legs of the patient with their feet in contact with the footplate so that the means for moving the footplate can be functionally actuated to provide a force to the feet of the patient and load the spine. As such, the means for moving the footplate with respect to the base member can be described in greater detail.

In one embodiment, the footplate is at least indirectly coupled to a force transferring member such as a transfer member 170 or a transfer plate. The coupling of the footplate and the force transferring member can be such that the footplate is enabled to move relative to the base member when a force is applied to the force transferring member. Accordingly, the force transferring member can be made of one or more components that cooperate to receive a force and then transfer the force to the footplate. However, the transferring member can also include various components such as those that transfer a tensile force from a spring so as to pull the footplate across the base or those that transfer a compressive force so as to push the footplate across the base.

In one embodiment, the support assembly is configured to include a footplate operatively coupled with a means for transferring a force to the footplate (“transfer means”). As such, a force exerted against the transfer means is transferred so as to move the footplate and/or the support assembly across the body side of the base. Various types of transfer means can be employed with the present invention so long as a force is thereby transferred so as to move the footplate across the base. Examples of transfer means includes spring systems, pulley systems, pneumatic systems, hydraulic systems, direct drive systems, indirect drive systems, chain drive systems, worm gears, other gear assemblies, and the like as well as a force transfer member or plate. The foregoing are also examples of the means for selectively moving the footplate.

Additionally, the spinal compression device can include at least one spring that is at least indirectly coupled to the force transferring member or transfer means. As such, the spring can be functionally coupled with the transfer means so that a force may be exerted by the spring against the transfer means. This coupling is such that when the spring receives a first force so as to compress or elongate the spring, a second force is thereby applied by the spring to the force transferring means by pulling or pushing. Accordingly, the spring can be arranged with respect to the footplate assembly and/or transfer means so as to receive and supply tensile or compressive forces. Alternatively, the spring can be at least indirectly coupled to the footplate so that a first force applied to a first end of the spring results in a second force being at least indirectly applied to the footplate by a second end of the spring. In any event, when a first force applied to a first end the at least one spring so as to compress or stretch the spring, a second force is applied to the transfer means by a second end of the spring.

In another embodiment, the spinal compression device can include a means for exerting a force on the spring so that the spring facilitates the movement of the footplate to the body side of the base. Accordingly, the means for exerting a first force to at least one spring functions so that the application of such a force can be propagated through various load biasing assembly components so as to cause movement of the footplate with respect to the base. Examples of means for exerting a force against a spring can include spring systems, pulley systems, pneumatic systems, hydraulic systems, direct drive systems, indirect drive systems, chain drive systems, worm gears, threaded gears, other gear assemblies, and the like as well as a drive plate or push plate. As such, actuation of such a means for exerting a force against a spring can operate to compress or elongate the spring.

Another embodiment includes a drive assembly that can be at least indirectly coupled with at least one spring. In any event, the drive assembly is operatively coupled with the spring in a manner that allows for forces to be exerted on the spring to either compress or stretch the spring. This enables the drive assembly to be configured so as to apply the first force to the spring when the drive assembly is actuated, where the drive assembly either directly or indirectly applies such a first force. Additionally, the drive assembly can be configured for pushing a push plate or drive plate against the spring to provide compressive forces to the force transfer member or transfer means. Alternatively, the drive assembly can be configured for pulling on a pull plate that is affixed to the spring so that an act of pulling elongates the spring, which provides tensile forces to the force transfer member or transfer means. In either of these processes, actuation of the drive assembly applies a force to the spring. Thus, actuation of the drive assembly can at least indirectly move the footplate across the body side of the base, which thereby enables compression of the spine of the patient.

In order for the drive assembly to apply a force to the spring, the spinal compression device can also include a means for actuating the drive assembly. Examples of drive assemblies and/or means for actuating the drive assembly can include motors, ceramic motors, pulley systems, pneumatic systems, hydraulic systems, direct drive systems, indirect drive systems, chain drive systems, worm gears, threaded gears, other gear assemblies, and the like as well as a crank assembly.

In an additional embodiment, the crank assembly can be at least indirectly coupled with a spring, which allows actuation of the crank assembly to apply the first force to the spring. Also, the crank assembly can be configured to be directly coupled with a drive mechanism or dive assembly that applies pressure to the spring. As such, the crank assembly can be fitted with a gear assembly that operates to actuate the drive mechanism when the crank assembly is rotated. Various types of gear assemblies can be included within the crank assembly. The crank assembly can be oriented so that its rotation directly, or through a belt, rotates a threaded gear to drive a plate for exerting the force against the spring. In any event, the crank assembly is actuated by a medical professional rotating a handle so as to rotate the crank.

Another embodiment of the present invention includes a method of compressing a spine of a patient during diagnostic imaging. The method includes an act of placing a patient in a supine position. Typically, when the spine is being imaged by x-ray, MRI, or CT-Scan, the patient is laid on a bed in the supine position. This is partly because the supine position is a comfortable position, and the patient can easily maintain this position for the duration of the imaging procedure. Additionally, most diagnostic imaging machinery is configured to take images of a patient in the supine position. The patient can also be placed in a prone or side position.

The method can also include an act of fitting the patient with a compression system. Accordingly, the compression system includes at least a footplate as part of a load biasing assembly and a restraint. As such, the fitting includes orienting the patient with respect to the compression system so that the footplate is positioned against a foot of the patient and the restraint engages the superior end of the patient such as the chest, shoulders or head of the patient.

The method can also include an act of exerting a force on a spring in the load biasing assembly. Accordingly, when the force is applied to the spring, the force is transferred therethrough by either compression or elongation so that a force is then applied to the footplate. As such, exerting the force against the spring causes movement of the footplate so as to compress the patient between the footplate and the restraint. In one embodiment, this is because when the footplate moves, the distance between the footplate and the restraint shortens, while the distance between the base of the load biasing assembly and the restraint stays the same.

Additionally, the load biasing assembly can include a means for exerting a force on the spring, as described herein, which facilitates such a movement of the footplate. In accordance therewith, the method can include an act of actuating the means for exerting the force on the spring. Such an act of actuation can include operating, rotating, driving, or otherwise causing the means to exert the force on the spring. Similar with other embodiments, the force received by the spring can be such that the spring at least indirectly loads the spine of the patient. As such, the loading can be placed on the spine of patient through a footplate.

In another embodiment, the method can include an act of actuating a drive assembly to apply a force to at least one spring. As such, a force is exerted by the at least one spring at least indirectly exerts a force against the footplate. By actuating the drive assembly, the drive assembly moves a component of the load biasing assembly so that it places a force on the spring, where the force can be either a compressive force or a tensile force. In some embodiments, actuation of the drive assembly can be performed by manually operating a crank assembly. Alternatively, the actuation can be by a removable motor that operates the drive assembly. In any event, the actuation results in a drive plate or other component exerting the force against the spring that is transferred to the footplate so as to compress the patient's spine.

Additionally, the method of compressing a spine of a patient during diagnostic imaging can be performed in a manner to include an act of compressing the spine of the patient in response to a load being placed thereon. Accordingly, compression of the spine can result in the imaged spine being shorter in length than the spine when in the supine position without being loaded. This is because the spine can be in a relaxed state while in the supine position, which can lengthen the spine compared to an upright or standing position. As such, providing compressive forces to the spine can cause the spine to shorten so that spaces, gaps, or other regions can become more compressed. This shortening during imaging can provide a medical professional with images that more correctly represent the spine during normal activities other than being supine.

Accordingly, the method can include an act of temporarily shortening the spine. Actual shortening of the spine can simulate the natural result of bearing weight. For example, sometimes a patient may feel more pain while standing or otherwise bearing weight on the spine. Imaging the spine in a supine position without the addition of a compressive load may result in images that do not accurately depict the status of the spine and associated tissue. As such, these images can result in the medical professional not being capable of identifying the cause of the pain. However, axially loading and/or shortening the spine during any diagnostic imaging can provide the medical practitioner with images that illustrate the compressed spine so that a more correct diagnosis can be obtained. As a result, the medical practitioner may be capable of making a diagnosis that would otherwise not be observable in images of the patient in a non-compressed and/or non-shortened supine position.

In another embodiment, the method can include maintaining a compressive force on the spine after the spine has compressed and shortened. As such, the load biasing assembly can be configured so that a compressive force or load is maintained against the patient even after the spine has compressed and shortened. In traditional spinal compressive devices, a spine can compress and shorten in response to the load, but the load or compressive force is not maintained because the device does not take into account such shortening. On the other hand, the spinal compression device of the instant invention can maintain a load on the spine even after the spine has shortened.

A load can be maintained on a shortened spine because of the nature of the spring. Accordingly, the at least one spring within the load biasing assembly can cooperate with the force that is applied thereto in a manner that continually transfers a load to a mechanism for facilitating movement of the footplate, even after the spine is shortened. This is because the spring continually either presses or pulls in an attempt to reach its initial condition. As such, when a force acts on the spring to either compress or stretch the spring, the spring shortens or lengthens during the transfer of the force. Accordingly, after the spine shortens, the spring can still exert some force before it is relaxed back to its original un-loaded length in the initial condition. While the load after spinal shortening can be less in comparison to the load on the pre-shortened spine, the spring may still maintain the compressive force and some load on the spine. In some instances, even after the spine has shortened, substantially the same force can still be applied through the footplate. As a result, a medical practitioner can image the spine while it is compressed and still maintaining a load. This advancement in. spinal imaging can provide the medical practitioner with images that depict a shortened spine in response to a load where some load is still being impinged on the spine.

In the above described embodiment of compression system 110, the crank is used to apply a predefined compressive load force to the patient. If desired and able, the operator can continually monitor and adjust the load during the imaging process. However, during many imaging procedures, the operator will not be able to access the patient or compression system 110. Thus in some alternative embodiments, automatic drive systems can be used that continually monitor and adjust the compressive load force on the patient so that the compressive load force is maintained within a desired range.

One of the challenges with these systems, however, is designing them so that they do not interfere with the diagnostic imaging machine. For example, a conventional motor could be mounted on base 122 so as to directly dive footplate 120 or springs 210. Load sensors can be coupled with the system so that the motor maintains the desired load force during the imaging. However, conventional motors could either interfere with or may not operate under the high magnetic fields produced by machines that create MRIs. However, piezo ceramic motors can be used without interfering with an MRI. In yet other embodiments, the drive mechanism could be remotely positioned. For example, pneumatic or hydraulic drive systems could be positioned remote from base 122 while hoses extend from the remove drive system to base 122. Again, the remote drive system can be used in association with load sensors to maintain the desired compressive load force on the patient during the diagnostic imaging. It yet other embodiments, the motor can be shielded, such as with lead, from the MRI.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A spine compression device comprising:
a base;
a footplate movably coupled with the base;
at least one spring being at least indirectly coupled with the footplate; and
a drive assembly configured to selectively apply a compressive or tensioning force to the at least one spring so that the spring applies a resilient force to the footplate, thereby causing the footplate to move relative to the base.

2. The spine compression device as recited in claim 1, further comprising a support plate having a top surface and an opposing bottom surface each extending between a first end and an opposing second end, the bottom surface of the support plate being slidably disposed on the base, the footplate being coupled to the support plate and upstanding from the top surface thereof.

3. The spine compression device as recited in claim 2, wherein the at least one spring is coupled with the support plate.

4. The spine compression devices as recited in claim 3, further comprising a transition member disposed on the bottom surface of the support plate, the at least one spring biasing against the transition member.

5. The spine compression device as recited in claim 4, further comprising:
a drive block movable relative to the base;
a threaded drive shaft extending between the drive block and the transfer member, the drive block being threadedly coupled with drive shaft;
the at least one spring extending between the drive block and the transfer member; and
the drive assembly comprising a crank assembly mounted on the base and coupled with the threaded drive shaft, whereby operation of the crank assembly facilitates rotation of the drive shaft which moves the drive block along. the drive shaft and biases the at least one spring against the transition member.

6. The spine compression device as recited in claim 5, further comprising a guide shaft extending between the drive block and the transition member, the at least one spring encircling the guide shaft.

7. The spine compression device as recited in claim 1, wherein the drive assembly comprises a hand operated crank assembly mounted on the base and coupled with the at least one spring.

8. The spine compression device as recited in claim 2, further comprising a pair of braces secured to the footplate and the support plate at the opposing sides thereof, the braces extending substantially orthogonal to the footplate.

9. The spine compression device as recited in claim 1, wherein the at least one spring comprises a plurality of springs, each of the plurality of spring being pre-compressed.

10. The spine compression device as recited in claim 1, further comprising:
a restraint adapted for engaging a superior end of a patient; and
a pair of elongated straps extending between the base and the harness.

11. The spine compression device as recited in claim 10, wherein the restraint comprises a harness adapted for mounting on the thorax of a patient.

12. The spine compression device as recited in claim 10, wherein the base comprises a table.

13. The spine compression device as recited in claim 12, further comprising a restraint adapted for engaging a superior end of a patient, the restraint being secured to the table.

14. The spine compression device as recited in claim 1, wherein the spring comprises a coiled spring.

15. A spine compression device comprising:
a base having a pair of opposing sides extending between a first end and an opposing second end;
a support assembly comprising a support plate movably disposed on the base and a footplate upstanding on the support plate;
a first spring coupled with the support assembly; and
means for exerting a force on the first spring such that the first spring applies a force to the support assembly and facilitates movement of the support assembly relative to the base.

16. A spine compression device as recited in claim 15, further comprising a restraint assembly connected to the base, the restrain assembly being configured to engage a superior end of a patient.

17. A spine compression device as recited in claim 16, wherein the restraint assembly comprises:
a harness adapted for mounting on the thorax of a patient, the harness having a pair of opposing sides; and
a pair of elongated straps, each strap being secured to a corresponding one of the opposing sides of the base and being adjustably secured to a corresponding one of the opposing sides of the harness.

18. A spine compression device as recited in claim 17, further comprising a pair of mounting arms upwardly projecting from the base above the support plate, each mounting arm being disposed on a corresponding one of the opposing sides of the base, each strap being secured to a corresponding one of the mounting arms.

19. A spine compression device as recited in claim 16, wherein the restraint assembly comprises:
a cap adapted for mounting on the head of a patient; and
a pair of elongated straps extending from the cap to the base.

20. A spine compression device as recited in claim 15, wherein the base comprises a table, a restraint being secured to the table, the restraint being adapted to engage a superior end of a patient.

21. A spine compression device as recited in claim 15, further comprising at least one load cell mounted on the foot plate.

22. A spine compression device as recited in claim 15, wherein the means for exerting a force on the first spring comprises a crank assembly coupled with the base.

23. A spine compression device as recited in claim 22, further comprising:
a threaded drive shaft coupled with the crank assembly; and
a drive block threadedly engaged with the drive shaft such that rotation of the drive shaft causes the drive block to move along the drive shaft, the first spring extending between the drive block and the support assembly.

24. The spine compression device as recited in claim 23, further comprising a guide shaft extending between the drive block, the first spring encircling the guide shaft.

25. A spine compression device as recited in claim 15, wherein the means for exerting a force on the first spring comprises a motor coupled with the base.

26. A spine compression device comprising:
a base having a first end and an opposing second end with a pair of opposing sides extending therebetween;
a footplate mounted on the base and being movable relative to the base;
means for selectively moving the footplate relative to the base;
a restraint adapted engage a superior end of a patient; and
a pair of elongated straps extending between the restraint and the base.

27. A spine compression device as recited in claim 26, wherein the restraint comprises a harness adapted to engage the thorax of a patient.

28. A spine compression device as recited in claim 27, further comprises:
the harness having a pair of opposing sides; and
each of the pair of elongated straps being secured to a corresponding one of the opposing sides of the base and being adjustably secured to a corresponding one of the opposing sides of the harness.

29. A spine compression device as recited in claim 26, further comprising a pair of mounting arms upwardly projecting from the base, each mounting arm being disposed on a corresponding one of the opposing sides of the base, each strap being secured to a corresponding one of the of the mounting arms.

30. A spine compression device as recited in claim 26, further comprising a support assembly, the support assembly comprising a support plate movably disposed on the base and the footplate secured to and upstanding on the support plate.

31. A spine compression device as recited in claim 26, wherein the means for selectively moving the footplate relative to the base comprises a crank assembly coupled with the base.

32. A spine compression device as recited in claim 31, further comprising:
a threaded drive shaft coupied with crank assembly; and
a drive block threadedly engaged with the drive shaft such that rotation of the drive shaft causes the drive block to move along the drive shaft. a first spring extending between the drive block and the support assembly.

33. The spine compression device as recited in claim 32, further comprising a guide shaft extending from the drive block, the first spring encircling the guide shaft.

34. A spine compression device as recited in claim 26, wherein the means for selectively moving the footplate relative to the base comprises a motor coupled with the base.

35. A method of compressing a spine comprising:
fitting a patient with a compression system so that a footplate movably disposed on a base is positioned against the feet of the patient and a restraint engages a superior end of the patient; and
after the patient is fitted with the compression system and is disposed in a position with the legs of the patient straightened, manipulating the compression system so that a force resiliently biases the footplate against the feet of the patient so as to apply a compressive load on the patient that compresses the spine of the patient.

36. The method as recited in claim 35, further comprising taking a diagnostic image of the spine of the patient.

37. The method as recited in claim 36, wherein the step of taking the diagnostic image comprises taking an MRI image.

38. The method as recited in claim 35, further comprising taking a fluoroscope image of the spine of the patient.

39. The method as recited in claim 35, further comprising delivering a pain relieving agent to a location on the spine of the patient.

40. The method as recited in claim 35, further comprising delivering a local anesthetic, steroid, contrasting agent or saline solution to a location on the spine of the patient.

41. The method as recited in claim 35, wherein the step of fitting the patient comprises securing the restraint to a head or thorax of a patient.

42. The method as recited in claim 35, wherein the restraint comprises a harness adapted to engage a thorax of a patient, the step of fitting the patient comprising:
securing the harness to the patient;
laying the patient on a table on which the base and footplate are supported, the patient being positioned on the table so that the feet of the patient are positioned against the footplate with the legs of the patient straightened; and
securing a pair of straps extending from the base to the harness.

43. The method as recited in claim 35, wherein the base comprises a table, the step of fitting the patient comprising laying the patient on the table so that the restraint that is secured to the table engages the superior end of the patient.

44. The method as recited in claim 35, wherein the step of manipulating the compression system so that the force resiliently biases the footplate against the feet of the patient comprises applying a force to a spring so that the spring biases the footplate against the feet of the patient, the spring being compressed by the force so as to provide the resilient biasing.

45. The method as recited in claim 44, wherein the step of applying the force to the spring comprises rotating a crank assembly.

46. The method as recited in claim 44, wherein the step of applying the force to the spring comprises activating a motor.

47. The method as recited in claim 35, further comprising measuring the compressive load on the patient prior to taking the diagnostic image.

48. The method as recited in claim 35, wherein the force that resiliently biases the footplate against the feet of the patient is not produced by a pneumatic pump.

49. A method of compressing a spine comprising:
fitting a patient with a compression system so that a footplate movably disposed on a base is positioned against the feet of the patient and a restraint engages a superior end of the patient;
manipulating the compression system so that a force biases the footplate against the feet of the patient so as to apply a compressive load on the patient that compresses the spine of the patient; and
delivering a pain relieving agent to a location on the spine of the patient.
